(19) United States
(12) Patent Application Publication
(10) Pub. No.: US 2017/0209088 A1
(43) Pub. Date: Jul. 27, 2017

(54) MASSAGE MACHINE AND PHYSICAL CONDITION MANAGEMENT SYSTEM

(71) Applicant: FAMILY INADA CO., LTD., Osaka (JP)

(72) Inventors: Nichimu Inada, Osaka (JP); Shinya Ikeda, Osaka (JP); Nagatoshi Harada, Osaka (JP); Yuichi Nakata, Tottori (JP)

(21) Appl. No.: 15/482,396

(22) Filed: Apr. 7, 2017

Related U.S. Application Data
(63) Continuation of application No. PCT/JP2015/075301, filed on Sep. 7, 2015.

(30) Foreign Application Priority Data
Oct. 7, 2014 (JP) 2014-206516

(51) Int. Cl. A61B 5/00; A61B 5/145; A61H 7/00; A61B 5/024

(52) U.S. Cl. CPC A61B 5/4848; A61B 5/024; A61B 5/14507; A61B 5/14546; A61H 7/001; A61H 2230/065; A61H 2205/081

(57) ABSTRACT

A massage machine includes a backrest and a seat, and further includes a treatment unit configured to perform a treatment with respect to a subject; a measurement unit configured to measure beat rates of the subject or an amylase activity in saliva of the subject before and after a predetermined time; and an evaluation portion. The evaluation portion performs an evaluation of the treatment based on a first index indicating a parasympathetic nervous function activity of the subject and a second index indicating a sympathetic nervous function activity of the subject. The first index and the second index are obtained based on the beat rates or the amylase activity before and after the predetermined time.

### RELATED APPLICATION

This is a continuation of PCT/JP2015/075301 filed on Sep. 7, 2015 claiming Paris Convention Priority based on Japanese Patent Application No. 2014-206516 filed on Oct. 7, 2014, the contents of these applications of which, including the specifications, claims and drawings, are incorporated herein by reference in their entirety.

### TECHNICAL FIELD

The present invention relates to a massage machine and a physical condition management system.

### BACKGROUND ART

Autonomic nerves which act independently of the will of a person include sympathetic nerves and parasympathetic nerves. When activity of a sympathetic nervous function is high and activity of a parasympathetic nervous function is low, a person is considered to be in a stressed state, and when the activity of the sympathetic nervous function is low and the activity of the parasympathetic nervous function is high, the person is considered to be in a relaxed state. In addition, an apparatus which determines the degree of stress based on the activity of the sympathetic nervous function and the parasympathetic nervous function by utilizing the above-described states of the nervous functions has been proposed (see Japanese Patent Application Publication No. H10-137228).

### SUMMARY OF INVENTION

A massage machine includes a backrest and a seat, and further includes a treatment unit configured to perform a treatment with respect to a subject; a measurement unit configured to measure beat rates of the subject or an amylase activity in saliva of the subject before and after a predetermined time; and an evaluation portion. The evaluation portion performs an evaluation of the treatment based on a first index indicating a parasympathetic nervous function activity of the subject and a second index indicating a sympathetic nervous function activity of the subject. The first index and the second index are obtained based on the beat rates or the amylase activity before and after the predetermined time.

### BRIEF DESCRIPTION OF DRAWINGS

FIG. 1 is a schematic view of the configuration of a physical condition management system.

FIG. 2 is a view illustrating a flow of beat rate acquisition control.

FIG. 3 is a view illustrating a flow of treatment evaluation processing.

FIG. 4 is a view illustrating a relationship between each index and an evaluation.

FIG. 5 is a view illustrating a flow of period evaluation processing.

### DESCRIPTION OF EMBODIMENTS

### <Configuration of Physical Condition Management System>

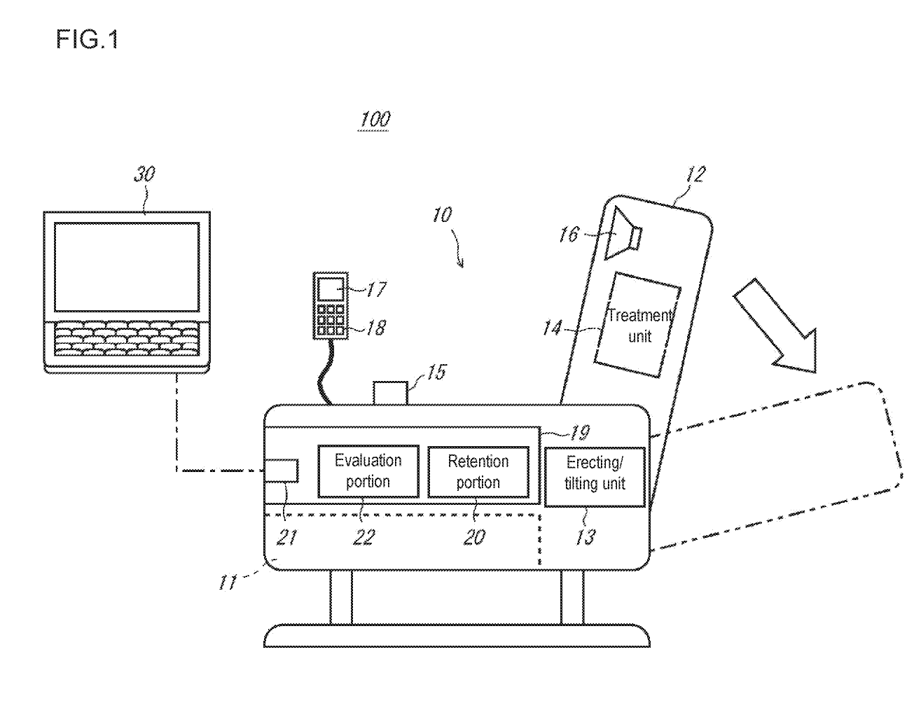

Hereinafter, a physical condition management system 100 according to an embodiment of the present invention will be described. First, the configuration of the physical condition management system 100 will be described. FIG. 1 is a schematic view of the configuration of the physical condition management system 100. As illustrated in FIG. 1, the physical condition management system 100 includes a massage machine 10 and external equipment 30.

The massage machine 10 has a seat portion 11 in which a treatment subject takes a seat, a backrest 12 which is disposed at the rear of the seat portion 11. An erecting/tilting unit 13 which causes the backrest 12 to be erected and tilted with respect to the seat portion 11. In other words, the backrest 12 is movable between a tilted position and an erected position with respect to the seat portion 11. A treatment unit 14 which performs treatment (massage) with respect to the treatment subject, a measurement unit (beat rate measurement unit) 15 which measures a beat rate of the treatment subject, a voice output portion 16 which outputs a voice, a display portion 17 which displays predetermined information, an input portion 18 through which the treatment subject inputs the predetermined information, and a control unit 19 which controls the massage machine 10 in its entirety.

Moreover, the control unit 19 has a retention portion 20 which retains various types of data, a data output portion 21 which outputs the data retained in the retention portion 20 to the external equipment 30, and an evaluation portion 22 which is functionally configured. The control unit 19 is electrically connected to the erecting/tilting unit 13, the treatment unit 14, the measurement unit 15, the voice output portion 16, and the display portion 17. The control unit 19 performs various types of control by transmitting a control signal to these units and portions. In addition, the control unit 19 is electrically connected to the input portion 18. The control unit 19 can acquire the predetermined information through this input portion 18 and the measurement unit 15.

### <Acquisition of Beat Rate>

Next, the contents of control performed by the control unit 19 will be described. The control unit 19 acquires beat rates of the treatment subject before treatment and after treatment. Based on the acquired beat rates, the control unit 19 evaluates whether or not physical condition of the treatment subject is improved after the treatment compared to before the treatment. Here, beat rate acquisition control for acquiring a beat rate will be described. The term “beat rate” denotes a heart rate or a pulse rate of the treatment subject. In the present embodiment, the pulse rate is acquired as the beat rate from a finger of the treatment subject. However, the pulse rate or the heart rate may be acquired from a different site.

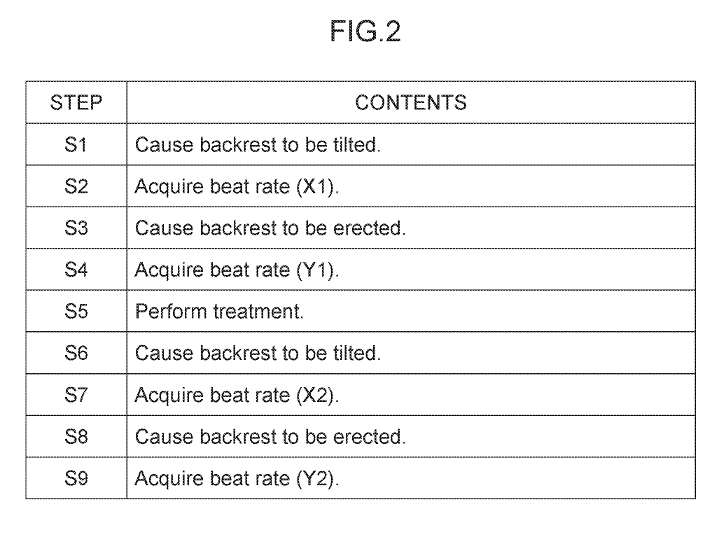

FIG. 2 is a view illustrating a flow of the beat rate acquisition control. When the control starts, the control unit 19 causes the backrest 12 to be tilted by using the erecting/tilting unit 13 (Step S1) such that the treatment subject is in a lying state (decubitus state). As an example, the angle of the backrest 12 in the decubitus state is an angle between a state parallel to the ground (horizontal level) and a state erected 30 degrees from the horizontal level (angle to the ground ranges from zero degrees to 30 degrees). In addition, at the same time the backrest 12 starts being tilted, the control unit 19 starts measuring the beat rate of the treatment subject by using the measurement unit 15. Before the backrest 12 starts being tilted, voice guidance such as “please attach a measuring instrument to a finger” and “reclining backrest will be tilted” may be output from the voice output portion 16. In addition, in a case where the backrest 12 cannot be set to the above-referenced angle, the treatment subject may directly take a posture corresponding to the above-referenced angle.

Subsequently, the control unit 19 acquires a beat rate of the treatment subject for a predetermined time (for example, for 30 seconds) through the measurement unit 15 (Step S2). In this case, the control unit 19 may cause the voice output portion 16 to output voice guidance such as “measurement cannot be normally performed when you sit up or operate the reclining backrest during the measurement”. The control unit 19 causes the retention portion 20 to retain the beat rate (pretherapeutic decubitus beat rate X1) acquired herein. In order to acquire stable data, instead of starting acquiring the beat rate immediately after the backrest 12 is tilted, it is desirable to start acquiring the beat rate after a predetermined time (for example, after 90 seconds) from when the backrest 12 is tilted.

Subsequently, the control unit 19 causes the backrest 12 to be erected by using the erecting/tilting unit 13 (Step S3) such that the treatment subject is in a seated state (sitting state). As an example, the angle of the backrest 12 in the sitting state is an angle between a state perpendicular to the ground and a state leaned back 30 degrees from the perpendicular line (angle to the ground ranges from 60 degrees to 90 degrees). When the backrest 12 is erected, the control unit 19 may cause the voice output portion 16 to output voice guidance such as “the reclining backrest will be erected”. The backrest 12 may start being erected immediately after the beat rate is measured, or the backrest 12 may start being erected after a predetermined time (for example, after 30 seconds) from when acquiring the beat rate is completed. In addition, in a case where the backrest 12 cannot be set to the above-referenced angle, the treatment subject may directly take a posture corresponding to the above-referenced angle.

Subsequently, the control unit 19 acquires a beat rate of the treatment subject for a predetermined time (for example, for 30 seconds) through the measurement unit 15 (Step S4). The control unit 19 causes the retention portion 20 to retain the beat rate (pretherapeutic sitting beat rate Y1) acquired herein. In order to acquire stable data, instead of starting acquiring the beat rate immediately after the backrest 12 is erected, it is desirable to start acquiring the beat rate after a predetermined time from when erecting the backrest 12 ends (for example, after 90 seconds from when the backrest 12 starts being erected). In addition, after the beat rate is acquired, the control unit 19 ends measuring the beat rate by using the measurement unit 15.

Subsequently, the control unit 19 causes the treatment unit 14 to perform treatment with respect to the treatment subject (Step S5). Before the treatment starts, the control unit 19 may cause the voice output portion 16 to output voice guidance such as “please detach the measuring instrument from the finger”. In addition, in a case where the backrest 12 is tilted at the time the treatment ends, the control unit 19 causes the backrest 12 to be temporarily erected by using the erecting/tilting unit 13. When the backrest 12 is temporarily erected, the treatment subject can easily perform work of attaching a sensor of the measurement unit 15 to a predetermined site (in the present embodiment, a finger).

Subsequently, the control unit 19 causes the backrest 12 to be tilted by using the erecting/tilting unit 13 (Step S6) such that the treatment subject is in the decubitus state. In this case, the control unit 19 may cause the voice output portion 16 to output voice guidance such as “the reclining backrest will be tilted”. At the same time the backrest 12 starts being tilted, the control unit 19 starts measuring the beat rate of the treatment subject by using the measurement unit 15.

Subsequently, the control unit 19 acquires a beat rate of the treatment subject for a predetermined time (for example, for 30 seconds) through the measurement unit 15 (Step S7). The control unit 19 causes the retention portion 20 to retain the beat rate (posttherapeutic decubitus beat rate X2) acquired herein. In order to acquire stable data, instead of starting acquiring the beat rate immediately after the backrest 12 is tilted, it is desirable to start acquiring the beat rate after a predetermined time from when tilting the backrest 12 ends (for example, after 90 seconds).

Subsequently, the control unit 19 causes the backrest 12 to be erected by using the erecting/tilting unit 13 (Step S8) such that the treatment subject is in the sitting state. In this case, the control unit 19 may cause the voice output portion 16 to output voice guidance such as “the reclining backrest will be erected”. The backrest 12 may start being erected immediately after acquiring the beat rate ends, or the backrest 12 may start being erected after a predetermined time (for example, after 30 seconds) from when acquiring the beat rate is completed.

Subsequently, the control unit 19 acquires a beat rate of the treatment subject through the measurement unit 15 (Step S9). The control unit 19 causes the retention portion 20 to retain the beat rate (posttherapeutic sitting beat rate Y2) acquired herein. In order to acquire stable data, instead of starting acquiring the beat rate immediately after the backrest 12 is erected, it is desirable to start acquiring the beat rate after a predetermined time from when erecting the backrest 12 ends (for example, after 90 seconds from when the backrest 12 starts being erected).

Hereinbefore, the beat rate acquisition control has been described. Through Steps S1 to S9 described above, the retention portion 20 retains the pretherapeutic decubitus beat rate X1 which is a beat rate of the treatment subject before the treatment in a state where the backrest 12 is tilted, the pretherapeutic sitting beat rate Y1 which is a beat rate of the treatment subject before the treatment in a state where the backrest 12 is erected, the posttherapeutic decubitus beat rate X2 which is a beat rate of the treatment subject after the treatment in a state where the backrest 12 is tilted, and the posttherapeutic sitting beat rate Y2 which is a beat rate of the treatment subject after the treatment in a state where the backrest 12 is erected.

<Evaluation of Improvement of Physical Condition after Treatment Compared to Before Treatment>

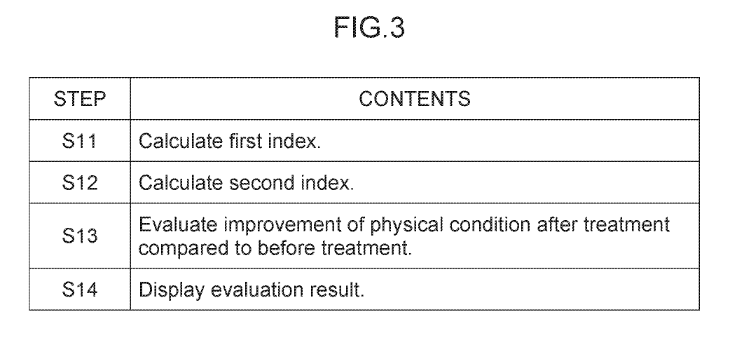

Next, treatment evaluation processing for evaluating whether or not the physical condition of the treatment subject is improved after the treatment compared to before the treatment will be described. FIG. 3 is a view illustrating a flow of the treatment evaluation processing. The evaluation portion 22 of the control unit 19 evaluates whether or not the physical condition of the treatment subject is improved after the treatment compared to before the treatment based on each of the beat rates X1, Y1, X2, and Y2 acquired through the beat rate acquisition control.

First, the evaluation portion 22 calculates a first index (Step S11). The first index is an index indicating variation of activity of a parasympathetic nervous function of the treatment subject after the treatment compared to before the treatment. The first index can be calculated through the following expression. That is, the first index is a value obtained by subtracting the posttherapeutic decubitus beat rate X2 from the pretherapeutic decubitus beat rate X1. In the decubitus state, in a case where the beat rate of the treatment subject has decreased after the treatment compared to before the treatment, it is considered that the parasympathetic nervous function is active, and the first index becomes positive.

First index=X1−X2

Fluctuation of an autonomic nervous function is indicated as fluctuation of the beat rate. However, the beat rate in a decubitus resting state is greatly influenced by the activity of the parasympathetic nervous function but is not much influenced by activity of a sympathetic nervous function. Therefore, in the first index which is calculated by using only the beat rate in the decubitus resting state, the influence of the activity of the sympathetic nervous function is substantially eliminated, and thus, the first index can be utilized as an index indicating the variation of the activity of the parasympathetic nervous function of the treatment subject after the treatment compared to before the treatment.

Subsequently, the evaluation portion 22 calculates a second index (Step S12). The second index is an index indicating variation of the activity of the sympathetic nervous function of the treatment subject after the treatment compared to before the treatment. The second index can be calculated through the following expression. That is, the second index is a value obtained by causing a value obtained by subtracting the pretherapeutic decubitus beat rate X1 from the pretherapeutic sitting beat rate Y1 to be further subtracted from a value obtained by subtracting the posttherapeutic decubitus beat rate X2 from the posttherapeutic sitting beat rate Y2. In a case where the difference between the beat rates of the treatment subject in the sitting state and the decubitus state is greater after the treatment compared to before the treatment, it is considered that the sympathetic nervous function is active, and the second index becomes positive. More specifically, the second index is an index indicating variation of activity of a sympathetic nerves β receptor system function. The sympathetic nerves β receptor system function influences the beat rate of an organ such as the heart.

Second index=(Y2−X2)−(Y1−X1)

As described above, the beat rate in the decubitus resting state is greatly influenced by the activity of the parasympathetic nervous function but is not much influenced by the activity of the sympathetic nervous function. In contrast, the beat rate in a sitting resting state is greatly influenced by both the activity of the parasympathetic nervous function and the activity of the sympathetic nervous function. In addition, the activity of the parasympathetic nervous function changes little due to a change of the body posture. Therefore, it is possible to consider that the difference between the beat rates of the treatment subject in the sitting resting state and the decubitus resting state indicates the activity of the sympathetic nervous function. Thus, the second index which is the difference of the activity of the sympathetic nervous function after the treatment compared to before the treatment can be used as an index indicating the variation of the activity of the sympathetic nervous function of the treatment subject after the treatment compared to before the treatment.

In the related art, indexes related to sympathetic function nerves and parasympathetic nerves function have been calculated through complicated calculation formulas. In contrast, in the present embodiment, by using the beat rates of the treatment subject in different postures such as the sitting state and the decubitus state of the treatment subject, it is possible to calculate indexes indicating variation of the activity of the sympathetic nerves function and the parasympathetic nervous function by using quite simple calculation formulas as described above.

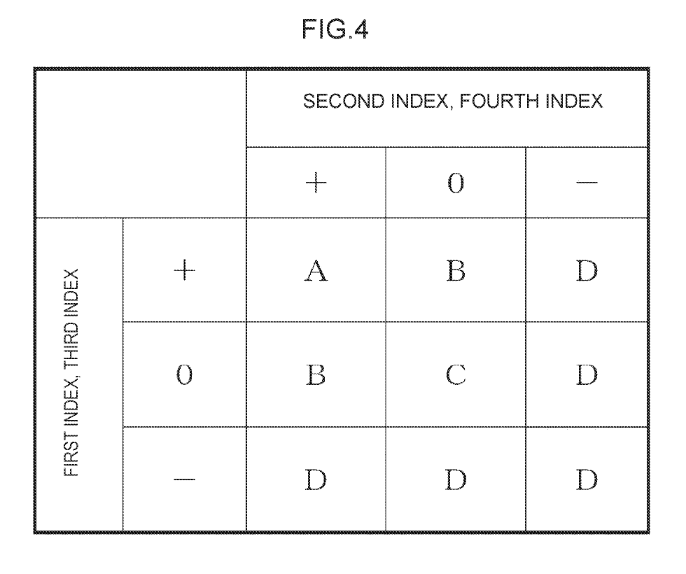

Subsequently, the evaluation portion 22 evaluates whether or not the physical condition of the treatment subject is improved after the treatment compared to before the treatment based on the first index and the second index (Step S13). FIG. 4 is a view illustrating a relationship between each index and an evaluation. As illustrated in FIG. 4, in the present embodiment, evaluations are performed in four categories of A, B, C, and D in descending order of the degree of improvement of the physical condition of the treatment subject.

When both the first index and the second index are positive, an evaluation is performed so as to confirm that the physical condition of the treatment subject is improved after the treatment compared to before the treatment, and the evaluation is categorized as “A”. In addition, even when one of the first index and the second index is positive and the other is zero, an evaluation is performed so as to confirm that the physical condition of the treatment subject is improved after the treatment compared to before the treatment. However, since the degree of improvement is smaller than that in a case of being categorized as A, the evaluation is categorized as “B”. In addition, when both the first index and the second index are zero, an evaluation is performed so as to confirm that there is no change in the physical condition of the treatment subject, and the evaluation is categorized as “C”. In addition, when at least one of the first index and the second index is negative, an evaluation is performed so as to confirm that the physical condition of the treatment subject is not improved or is deteriorated after the treatment compared to before the treatment, and the evaluation is categorized as “D”.

In regard to the first index, when a calculated value ranges from −1 to +1 (or greater than −1 and smaller than +1), the value may be considered to be zero. In regard to the second index, when a calculated value ranges from −0.5 to +0.5 (or greater than −0.5 and smaller than +0.5), the value may be considered to be zero. In this manner, the reason the error range of the first index is greater than the error range of the second index is that the beat rate is further influenced by the parasympathetic nervous function.

As described above, in the related art, the activity of the sympathetic nerves function and the activity of the parasympathetic nervous function are considered to be contrary to each other. However, there are cases where both the activity of the parasympathetic nerves function and the activity of the sympathetic nervous function practically increase. In other words, there are cases where the autonomic nervous function increases in its entirety. Considering that the purpose of a massage is to not only relieve stress of the treatment subject but also is to vitalize the body of the treatment, according to the present embodiment in which the physical condition of the treatment subject is improved when the activity increases in both the sympathetic nerves which function when the body is vitalized and the parasympathetic nerves which function when the body is relaxed, whether or not the physical condition of the treatment subject is improved through the treatment is exactly evaluated.

Subsequently, the evaluation portion 22 causes the display portion 17 to display the evaluation result (Step S14). As a display method, the categories (A, B, C, and D) may be displayed without any change. However, displaying may be performed through a different display method. For example, displaying may be performed by using a sign or a numerical value corresponding to each of the categories. The evaluation portion 22 causes the retention portion 20 to retain the evaluation result (evaluation data) obtained through the treatment evaluation processing.

Hereinbefore, description has been given regarding a case of using both the first index and the second index so as to evaluate whether or not the physical condition of the treatment subject is improved after the treatment compared to before the treatment. However, the evaluation may be performed by using only the first index. For example, when the first index is positive, an evaluation may be performed so as to confirm that “the physical condition is improved”. When the first index is zero, an evaluation may be performed so as to confirm that “there is no change in the physical condition”. When the first index is negative, an evaluation may be performed so as to confirm that “the physical condition is not improved” or “the physical condition is deteriorated”.

<Evaluation of Improvement of Physical Condition Over Evaluation Period>

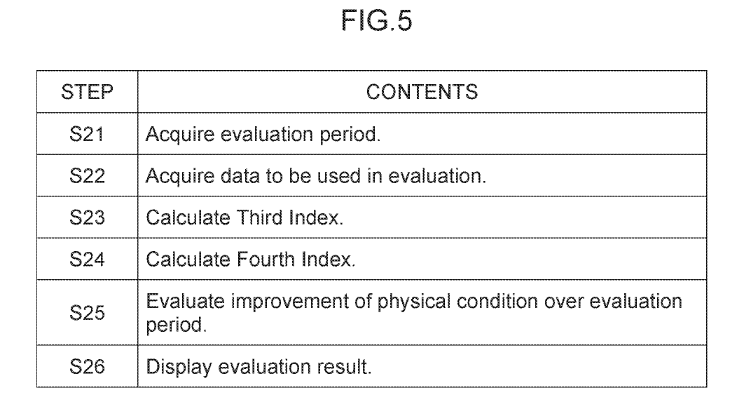

Next, description will be given regarding period evaluation processing of evaluating whether or not the physical condition of the treatment subject is improved over a predetermined evaluation period. FIG. 5 is a view illustrating a flow of the period evaluation processing. The evaluation portion 22 evaluates whether or not the physical condition of the treatment subject is improved over the predetermined evaluation period such as a week, a month, and a year designated by the treatment subject.

First, the evaluation portion 22 acquires the evaluation period (Step S21). The evaluation period is a period over which whether or not the physical condition of the treatment subject is improved becomes an evaluation target. The evaluation period is decided by the treatment subject and is input by the treatment subject via the input portion 18.

Subsequently, the evaluation portion 22 acquires data used in an evaluation (Step S22). Specifically, a preperiod decubitus beat rate X3 which is a beat rate of the treatment subject before the evaluation period in a state where the backrest 12 is tilted, a preperiod sitting beat rate Y3 which is a beat rate of the treatment subject before the evaluation period in a state where the backrest 12 is erected, a postperiod decubitus beat rate X4 which is a beat rate of the treatment subject after the evaluation period in a state where the backrest 12 is tilted, and a postperiod sitting beat rate Y4 which is a beat rate of the treatment subject after the evaluation period in a state where the backrest 12 is erected are acquired.

The above-described term “before the evaluation period” includes immediately before and immediately after the evaluation period starts, and the term “after the evaluation period” includes immediately before and immediately after the evaluation period ends. In the present embodiment, the evaluation portion 22 acquires the pretherapeutic decubitus beat rate X1 and the pretherapeutic sitting beat rate Y1 in a first day over the evaluation period and the pretherapeutic decubitus beat rate X1 and the pretherapeutic sitting beat rate Y1 in a last day over the evaluation period, in physical condition data retained in the retention portion 20, which respectively become the preperiod decubitus beat rate X3, the preperiod sitting beat rate Y3, the postperiod decubitus beat rate X4, and the postperiod sitting beat rate Y4.

Subsequently, the evaluation portion 22 calculates a third index (Step S23). The third index is an index indicating the variation of the activity of the parasympathetic nervous function of the treatment subject over the evaluation period. The third index can be calculated through the following expression. That is, the third index is a value obtained by subtracting the postperiod decubitus beat rate X4 from the preperiod decubitus beat rate X3. In the decubitus state, in a case where the beat rate of the treatment subject has decreased after the evaluation period compared to before the evaluation period, it is considered that the parasympathetic nerves function is active, and the third index becomes positive.

Third index=X3−X4

Subsequently, the evaluation portion 22 calculates the forth index (Step S24). The fourth index is an index indicating the variation of the activity of the sympathetic nervous function of the treatment subject over the evaluation period. The fourth index can be calculated through the following expression. That is, the fourth index is a value obtained by causing a value obtained by subtracting the preperiod decubitus beat rate X3 from the preperiod sitting beat rate Y3 to be further subtracted from a value obtained by subtracting the postperiod decubitus beat rate X4 from the postperiod sitting beat rate Y4. In a case where the difference between the beat rates of the treatment subject in the sitting state and the decubitus state is greater after the evaluation period compared to before the evaluation period, it is considered that the sympathetic nerves function is active, and the fourth index becomes positive.

Fourth index=(Y4−X4)−(Y3−X3)

Subsequently, based on the third index and the fourth index, the evaluation portion 22 evaluate whether or not the physical condition of the treatment subject is improved over the evaluation period (Step S25). The evaluation method in the period evaluation processing is basically the same as the evaluation method in the above-described treatment evaluation processing. That is, as illustrated in FIG. 4, based on the values of the third index and the fourth index, evaluations are performed by using four categories of A, B, C, and D in descending order of the degree of improvement of the physical condition of the treatment subject.

Subsequently, the evaluation portion 22 causes the display portion 17 to display the evaluation result (Step S26). Accordingly, the treatment subject can check the evaluation result. Regarding the display method, similar to a case of the above-described treatment evaluation processing, the categories (A, B, C, and D) may be displayed without any change. However, displaying may be performed through a different display method. The evaluation portion 22 causes the retention portion 20 to retain the evaluation result (evaluation data) through the period evaluation processing, and each of the beat rates X3, Y3, X4, and Y4 used in the evaluation.

Hereinbefore, description has been given regarding a case of using both the third index and the fourth index so as to evaluate whether or not the physical condition of the treatment subject is improved over the predetermined evaluation period. However, the evaluation may be performed by using only the third index. For example, when the third index is positive, an evaluation may be performed so as to confirm that “the physical condition is improved”. When the third index is zero, an evaluation may be performed so as to confirm that “there is no change in the physical condition”. When the third index is negative, an evaluation may be performed so as to confirm that “the physical condition is not improved” or “the physical condition is deteriorated”.

### <Utilization of Physical Condition Data>

As described above, through the beat rate acquisition control, the treatment evaluation processing, and the period evaluation processing, the retention portion 20 retains each piece of the evaluation data and the physical condition data including each of the beat rates used in the evaluations. In a physical condition management system according to the present embodiment, the external equipment 30 can acquire the physical condition data from the retention portion 20 via the data output portion 21. The external equipment 30 may be a personal computer, a tablet terminal, or a smart phone for general use and may be a dedicated terminal.

In the external equipment 30, software which can perform processing such as graphical displaying of a part of data included in the physical condition data is installed. Therefore, the external equipment 30 can perform various types of processing based on the physical condition data. With reference to the result of the processing, the treatment subject can directly manage the physical condition. The data output portion 21 may output the physical condition data via a cable, may output the physical condition data via radio communication means such as infrared rays, or may output the physical condition data via a USB memory or the like.

### Another Embodiment

In the above-described physical condition management system 100, improvement of the physical condition of the treatment subject is evaluated based on the beat rate. However, improvement of the physical condition of the treatment subject may be evaluated based on different information other than the beat rate. For example, since the degree of stress of the treatment subject can be determined based on amylase activity in saliva of the treatment subject (amount of amylase in saliva), improvement of the physical condition of the treatment subject may be evaluated by using the amylase activity in saliva.

That is, in the above-described physical condition management system 100, the measurement unit 15 may be an amylase activity measurement unit which measures the amylase activity in saliva of the treatment subject, and the evaluation portion 22 may be configured to evaluate improvement of the physical condition of the treatment subject after the treatment compared to before the treatment based on the amylase activity in saliva of the treatment subject after the treatment compared to before the treatment. In addition, the evaluation portion 22 may be configured to evaluate improvement of the physical condition of the treatment subject over the evaluation period based on the amylase activity in saliva of the treatment subject before the evaluation period and after the evaluation period.

According to such a configuration, for example, the evaluation portion 22 can determine whether or not stress of the treatment subject is improved after the treatment compared to before the treatment, based on the amylase activity in saliva of the treatment subject. Therefore, it is possible to further evaluate whether or not the physical condition of the treatment subject is improved through treatment, based on the result thereof. In this manner, according to a technique of using the amylase activity in saliva of the treatment subject, for being different from a technique of using the beat rate of the treatment subject, it is possible to perform a more exact evaluation according to the conditions.

Hereinbefore, embodiments of the present invention are described. It should be understood that the invention is not limited to the above-described embodiment, but may be modified into various forms on the basis of the spirit of the invention. Additionally, the modifications are included in the scope of the invention.

What is claimed is:

1. A massage machine comprising a backrest and a seat, the massage machine further comprising:
a treatment unit configured to perform a treatment with respect to a subject;
a measurement unit configured to measure beat rates of the subject or an amylase activity in saliva of the subject before and after a predetermined time; and
an evaluation portion configured to perform an evaluation of the treatment based on a first index indicating a parasympathetic nervous function activity of the subject and a second index indicating a sympathetic nervous function activity of the subject, wherein the first index and the second index are obtained based on the beat rates or the amylase activity before and after the predetermined time.

2. The massage machine according to claim 1,
the evaluation portion is configured to determine that, when the first index increases and the second index increases, a physical condition of the subject is improved.

3. The massage machine according to claim 1,
the evaluation portion is configured to determine that, when the first index decreases and the second index decreases, a physical condition of the subject is not improved.

4. The massage machine according to claim 1, the backrest is movable between a tilted position and an erected position with respect to the seat, the first index is configured to be obtained when the backrest is at the tilted position.

5. The massage machine according to claim 1, the backrest is movable between a tilted position and an erected position with respect to the seat, the second index is configured to be obtained when the backrest is at the erected position.

6. The massage machine according to claim 4, the second index is configured to be obtained when the backrest is at the erected position.

7. The massage machine according to claim 1,
the beat rates include a first beat rate of the subject measured before the predetermined time and a second beat rate of the subject measured after the predetermined time, the first index and the second index are obtained based on the first beat rate and the second beat rate.

8. The massage machine according to claim 7,
the evaluation portion is configured to determine that, when the first index of before the treatment increases after the treatment and the second index before the treatment increases after the treatment, a physical condition of the subject is improved.

9. The massage machine according to claim 7,
the evaluation portion is configured to determine that, when the first index of before the treatment decreases after the treatment and the second index before the treatment decreases after the treatment, a physical condition of the subject is not improved.

10. The massage machine according to claim 7, the backrest is movable between a tilted position and an erected position with respect to the seat, the first index is configured to be obtained when the backrest is at the tilted position.

11. The massage machine according to claim 10, the evaluation portion is configured to determine that, when the first beat is smaller than the second beat, the first index increases, and that, when the first beat is larger than the second beat, the first index decreases.

12. The massage machine according to claim 7, the backrest is movable between a tilted position and an erected position with respect to the seat, the second index is configured to be obtained when the backrest is at the erected position.

13. The massage machine according to claim 12, the evaluation portion is configured to determine that, when the first beat is smaller than the second beat, the second index increases, and that, when the first beat is larger than the second beat, the second index decreases.

14. The massage machine according to claim 10, the second index is configured to be obtained when the backrest is at the erected position.

15. The massage machine according to claim 14,
the evaluation portion is configured to determine that, when the first beat of the first index or the second index is smaller than the second beat, the first index or the second index increases, and that, when the first beat is larger than the second beat, the first index or the second index decreases.

16. The massage machine according to claim 15,
the evaluation portion is configured to determine that, when the first index increases and the second index increases, a physical condition of the subject is improved.

17. The massage machine according to claim 15,
the evaluation portion is configured to determine that, when the first index decreases and the second index decreases, a physical condition of the subject is not improved.

18. The massage machine according to claim 1, the predetermined time is a time for the treatment.

19. The massage machine according to claim 1, further comprising:
a retention portion that retains an evaluation data of the evaluation and a measurement data obtained by the measurement unit; and
a data output portion that is configured to output the data retained in the retention portion to an external equipment.

20. A physical condition management system comprising:
the massage machine according to claim 19; and
the external equipment that performs a predetermined processing based on the data.
